SEQUENCE LISTING (1 sequence)

SEQ ID NO: 1 - DNA, 663 bp, Trichoderma longibrachiatum
  source 1..663: mol_type = genomic DNA
tggaagtaaaaatcgtaacaaggtctccgttggtgaaccagcggagggatcattaccgagtttacaactcccaaaccccaatgtgaacgttaccaatctgttgcctcggcgggattctcttgccccgggcgcgtcgcagccccggatcccatggcgcccgccggaggaccaactccaaactcttttttctctccgtcgcggctcccgtcgcggctctgtttcatttttgctctgagcctttctcggcgaccctagcgggcgtctcgaaaatgaatcaaaactttcaacaacggatctcttggttctggcatcgatgaagaacgcagcgaaatgcgataagtaatgtgaattgcagaattcagtgaatcatcgaatctttgaacgcacattgcgcccgccagtattctggcgggcatgcctgtccgagcgtcatttcaaccctcgaacccctccggggggtcggcgttggggatcggcccctcaccgggccgcccccgaaatacagtggcggtctcgccgcagcctctcctgcgcagtagtttgcacactcgcaccgggagcgcggcgcggccacagccgtaaaacaccccaaacttctgaaatgttgacctcggatcaggtaggaatacccgctgaacttaagcatatcaataagcggaggaa

(19) United States
(12) Patent Application Publication
(10) Pub. No.: US 2026/0357190 A1
(43) Pub. Date: Nov. 12, 2026

(54) TRICHODERMA LONGIBRACHIATUM STRAIN CAPABLE OF DEGRADING PETROLEUM HYDROCARBONS IN SALINE-ALKALI ENVIRONMENT, AND CULTURE AND APPLICATION THEREOF

(71) Applicants: Northwest Institute of Eco-Environment and Resources, Chinese Academy of Sciences, Lanzhou (CN); Institute of Geology and Geophysics, Chinese Academy of Sciences, Beijing (CN)

(72) Inventors: Yingqin WU, Lanzhou (CN); Yuanyuan SHAO, Lanzhou (CN); Xiaofeng MA, Lanzhou (CN); Wenyang WANG, Beijing (CN); Longmiao YUAN, Lanzhou (CN); Wenwen ZHANG, Lanzhou (CN); Zhongping LI, Lanzhou (CN); Lantian XING, Lanzhou (CN); Yanhong LIU, Lanzhou (CN); Yan LIU, Lanzhou (CN)

(21) Appl. No.: 19/466,476

(22) Filed: Feb. 1, 2026

(30) Foreign Application Priority Data
Nov. 28, 2025 (CN) 202511775046.5

(51) Int. Cl. C12N 1/145; B09C 1/10; C12R 1/885

(52) U.S. Cl. CPC C12N 1/145; B09C 1/10; C12R 2001/885

(57) ABSTRACT

A Trichoderma longibrachiatum strain, which has been deposited in China General Microbiological Culture Collection Center (CGMCC) on Sep. 5, 2025 with an accession number of CGMCC No. 42182. A culture of the Trichoderma longibrachiatum strain is also provided, which is a fungal seed culture, a fermentation broth, a cell paste of the Trichoderma longibrachiatum strain, or a soluble extract, a concentrate or a dried product obtained from the fermentation broth. An application of the Trichoderma longibrachiatum strain in the treatment of petroleum hydrocarbon-polluted environmental samples is further provided.

### REFERENCE TO AN ELECTRONIC SEQUENCE LISTING

The contents of the electronic sequence listing (SequenceListing.xml; Size: 2,698 bytes; and Date of Creation: Jan. 8, 2026) are herein incorporated by reference in its entirety.

### CROSS-REFERENCE TO RELATED APPLICATIONS

This application claims the benefit of priority from Chinese Patent Application No. 202511775046.5, filed on Nov. 28, 2025. The content of the aforementioned application, including any intervening amendments thereto, is incorporated herein by reference in its entirety.

### TECHNICAL FIELD

This application relates to microbial biotechnology, and more particularly to a Trichoderma longibrachiatum strain capable of degrading petroleum hydrocarbons in a saline-alkali environment, and a culture and application thereof.

### BACKGROUND

As an important strategic material, petroleum plays a critical role in the development of modern economy and industry. However, improper operations and leakage accidents during the petroleum extraction, transportation, production and processing will cause environmental pollution. Oil spills will increase the total organic carbon content in soil, which will result in the carbon-nitrogen ratio imbalance, and change physical and chemical properties of the soil, thereby affecting the soil enzyme activity. Moreover, the petroleum hydrocarbons will accumulate in the soil for a long time. N-alkanes will deposit into the subsurface sediment through geochemical processes, which will pose a threat to benthic organisms. Among aromatic hydrocarbons, polycyclic aromatic hydrocarbons (PAHs) are carcinogenic and difficult to degrade. They enter the human body through the food chain, threatening human life and health.

Microbial remediation depends on the natural metabolism and co-metabolism mechanisms of microorganisms to degrade petroleum hydrocarbons in the contaminated soil and convert them into non-polluting subjects, improving soil environmental conditions and making it more suitable for microbial survival. It is a cost-effective, low-toxic and environmentally-friendly petroleum pollution remediation method, which has gained widespread attention at home and abroad in recent years.

However, the growth and remediation performance of microorganisms are often limited by conditions such as pH and salinity. Fungi are potential petroleum hydrocarbon-degrading microorganisms because they have branched hyphae that can penetrate into the soil matrix more effectively than bacteria, fully contact with petroleum pollutants, and degrade white pollutants through the secretion of extracellular enzymes that are difficult to remove. At the same time, fungi have strong environmental adaptability and can grow under different extreme environmental conditions. Therefore, fungi have profound research significance and broad application prospects in the microbial remediation of petroleum-contaminated soil. An optimal pH value of soil microorganisms is often around 7.0, and a salinity is around 0.2%. High salinity and overly acidic and alkaline environments will inhibit the activity of microorganisms. Drought, lack of water, salt-alkali accumulation, and high soil pH and salinity characteristics have become the main obstacles to the microbial remediation of petroleum hydrocarbon pollution. Therefore, there is an urgent need for an efficient method to specifically address the petroleum pollution in saline-alkaline soil.

In view of this, the present disclosure is proposed herein.

### SUMMARY

An object of the present disclosure is to provide a Trichoderma longibrachiatum strain capable of degrading petroleum hydrocarbons in a saline-alkali environment, and a culture and application thereof, so as to address the petroleum pollution in saline-alkali soil regions.

The present disclosure is implemented through the following technical solutions.

In a first aspect, the present disclosure provides a Trichoderma longibrachiatum strain, wherein the Trichoderma longibrachiatum strain has been deposited in China General Microbiological Culture Collection Center (CGMCC) on Sep. 5, 2025 with an accession number of CGMCC No. 42182.

In a second aspect, the present disclosure provides a method of degrading petroleum hydrocarbons in a petroleum hydrocarbon-containing sample, comprising:
mixing the aforementioned Trichoderma longibrachiatum strain with the petroleum hydrocarbon-containing sample.

In a third aspect, the present disclosure provides a culture, wherein the culture is obtained through culture of the aforementioned Trichoderma longibrachiatum strain in a culture medium.

In a fourth aspect, the present disclosure provides a method of treating a petroleum hydrocarbon-polluted sample, comprising:

inoculating the aforementioned Trichoderma longibrachiatum strain into the petroleum hydrocarbon-polluted sample followed by culture.

Compared to the prior art, the present disclosure has the following beneficial effects.

The Trichoderma longibrachiatum strain provided herein has a highly-efficient degradation characterization of petroleum. Specifically, under conditions of 30 °C, 150 r/min, pH 7.5-7.8, salinity 0.9% and crude oil concentration 2 g/L, after 14 days of reaction, it can degrade 86.77% of total petroleum hydrocarbons. Among the total petroleum hydrocarbons, a degradation rate of n-alkanes is 91.5%. Degradation rates of pristane and phytane in isoprenoid alkanes are 85.4% and 81.9%, respectively. Degradation rates of phenanthrene, methylphenanthrene, dimethylphenanthrene and trimethylphenanthrene in polycyclic aromatic hydrocarbons are 97.3%, 91.5%, 93.2% and 93.7%, respectively. Therefore, the present disclosure provides a practical and effective solution to address the petroleum pollution in saline-alkali soil regions, which can efficiently degrade petroleum in saline-alkali soil regions. In addition, the Trichoderma longibrachiatum strain provided herein can also be used for the degradation of kitchen waste oils and fats, possessing a good application prospect.

### BRIEF DESCRIPTION OF THE DRAWINGS

In order to make the technical solutions of the embodiments of the present disclosure clearer, the accompanying drawings in the embodiments of the present disclosure will be briefly described below. It should be understood that the accompanying drawings below merely illustrate some embodiments of the present disclosure, and thus should not be construed as limitation to the present disclosure. Based on the accompanying drawings, other related accompanying drawings can be obtained by those of ordinary skill in the art without paying creative effort.

FIGS. 1a-1b show a microscopic photograph and a colonial morphology of Trichoderma longibrachiatum strain 39-2 after 3 days of incubation, where 1a: microscopic photograph; and 1b: colonial morphology;

FIG. 2 is a petroleum-degrading plate of the Trichoderma longibrachiatum strain 39-2 after 7 days of incubation;

FIG. 3 is a phylogenetic tree of the Trichoderma longibrachiatum strain 39-2;

FIGS. 4a-4b are gas chromatography-mass spectrometry (GC-MS) chromatograms of crude oil before and after degradation, where 4a: before degradation; and 4b: after degradation (Pr is pristane, and Ph is phytane);

FIGS. 5a-5b are GC-MS chromatograms of n-alkanes and isoprenoid alkanes before and after degradation, where 5a: before degradation; and 5b: after degradation; and

FIGS. 6a-6b are GC-MS chromatograms of phenanthrene and methylphenanthrene series before and after degradation, where 6a: before degradation; and 6b: after degradation.

### DETAILED DESCRIPTION OF EMBODIMENTS

References to the embodiments of the present disclosure will be provided in detail below, and one or more examples of them are described. The examples are merely illustrative, and not intended to limit this present disclosure. It should be noted that, obviously, various modifications and changes can be made by those ordinary skill in the art without departing from the scope and spirit of the present disclosure. For example, features described or illustrated as a part of an embodiment can be used in another embodiment to obtain a further embodiment.

In a first aspect, the present disclosure provides a Trichoderma longibrachiatum strain, which has been deposited in China General Microbiological Culture Collection Center (CGMCC) on Sep. 5, 2025 with an accession number of CGMCC No. 42182.

The present disclosure provides a highly-efficient petroleum-degrading strain from soil near the oil well platform of the Changqing oilfield in the Ordos basin, which is obtained through domestication and isolation with crude oil from the Changqing oilfield as the sole carbon source.

The biochemical and physicochemical characteristics of the Trichoderma longibrachiatum strain provided herein are described below. The hyphae are white and cottony in an initial stage, turning yellowish-green to dark green upon maturation due to the production of conidia. Positive results are observed for cellulose hydrolysis, starch hydrolysis, gelatin liquefaction, lipid production and hydrogen peroxide (H₂O₂) test, while negative result is observed for acid production. The colony appears white initially, later becoming yellowish-green, cottony and rapidly expanding.

The Trichoderma longibrachiatum strain provided herein degrades 86.77% of total petroleum hydrocarbons under conditions of high salinity and alkalinity. Among the total petroleum hydrocarbons, a degradation rate of n-alkanes is 91.5%. Degradation rates of pristane and phytane in isoprenoid alkanes are 85.4% and 81.9%, respectively. Additionally, the Trichoderma longibrachiatum strain 39-2 also exhibits higher degradation rates for aromatic hydrocarbon components, with degradation rates of phenanthrene, methylphenanthrene, dimethylphenanthrene and trimethylphenanthrene reaching 97.3%, 91.5%, 93.2% and 93.7%, respectively. The present disclosure enables the isolation and identification of a specific Trichoderma longibrachiatum strain suitable for a saline-alkali environment, thereby establishing a foundation for the remediation of petroleum pollution in the saline-alkaline environment.

In a second aspect, the present disclosure provides a method of degrading petroleum hydrocarbons in a petroleum hydrocarbon-containing sample, including mixing the aforementioned Trichoderma longibrachiatum strain with the petroleum hydrocarbon-containing sample.

In some embodiments, the Trichoderma longibrachiatum strain is capable of triggering degradation of total petroleum hydrocarbons, degradation of complex petroleum hydrocarbon compounds or a combination thereof.

In some embodiments, the petroleum hydrocarbon-containing sample is crude oil.

In some embodiments, the petroleum hydrocarbons are crude oil from the Changqing oilfield.

In some embodiments, a seed culture of the Trichoderma longibrachiatum strain is used and prepared. The preparation method is described below. 5 mL of 0.9% physiological saline solution is used to rinse filamentous fungal spores preserved on a slant into 100 mL of Czapek's medium to obtain a mixture solution. The mixture solution is incubated at 28 °C with shaking at 140 r/min for 36 hours to obtain the seed culture.

In some embodiments, the petroleum hydrocarbons include at least one of n-alkanes, isoprenoid alkanes and aromatic hydrocarbon compounds, where the isoprenoid alkanes are selected from the group consisting of pristane, phytane and a combination thereof, and the aromatic hydrocarbon compounds include, but are not limited to, polycyclic aromatic hydrocarbons (PAHs), such as phenanthrene, methylphenanthrene, dimethylphenanthrene and trimethylphenanthrene.

The Trichoderma longibrachiatum strain provided herein, under conditions of 30 °C, 150 r/min, pH 7.5-7.8, salinity 0.9% and crude oil concentration 2 g/L, after 14 days of degradation reaction, achieves degradation rates of phenanthrene, methylphenanthrene, dimethylphenanthrene and trimethylphenanthrene in the culture medium of 97.3%, 91.5%, 93.2% and 93.7%, respectively, thereby demonstrating a high degradation rate.

In a third aspect, the present disclosure provides a culture, where the culture is obtained through culture of the aforementioned Trichoderma longibrachiatum strain in a culture medium.

In some embodiments, the culture is a seed culture, a fermentation broth or a cell paste of the Trichoderma longibrachiatum strain, or a soluble extract, a concentrate or a dried product obtained from the fermentation broth. The cell paste is obtained through centrifugation or static settling. The dried product is selected from the group consisting of a spray-dried product, a freeze-dried product, a vacuum-dried product and a drum-dried product.

In some embodiments, the culture is in a form of a bacterial agent, which, for example, is prepared by diluting fermentation products.

In a fourth aspect, the present disclosure provides a method for treating a petroleum hydrocarbon-polluted sample, including inoculating the aforementioned Trichoderma longibrachiatum strain into the petroleum hydrocarbon-polluted sample followed by culture.

In some embodiments, the petroleum hydrocarbon-polluted sample is a petroleum-polluted environmental sample.

In order to make the objects, technical solutions and advantages of the present disclosure clearer, the technical solution in the embodiments of the present disclosure will be clearly and completely described. Unless otherwise specified, the experiments in the following embodiments are performed under conventional conditions or conditions recommended by the manufacturer, and the reagents or instruments are all commercially available.

The features and performance of the present disclosure will be further detailly described in combination with the embodiments.

### Embodiment 1

This embodiment provided an isolation and identification of the Trichoderma longibrachiatum strain 39-2, which were performed through the following steps.

Soil containing petroleum hydrocarbons at a depth of 5-10 cm was collected from the surrounding area of the Changqing oilfield. After removing stones and plant residues with tweezers, 5 g of the soil was weighed and added to a sterilized conical flask containing 200 mL of sterile water. The conical flask was then incubated on a shaker at 28 °C with shaking at 150 r/min for 12 hours. After shaking, the conical flask was allowed to stand for 1 hour to obtain a suspension. 5 mL of the suspension was transferred into 45 mL of enrichment culture medium, followed by incubation on a shaker at 28 °C with shaking at 160 r/min for 3 days to obtain a culture solution. 5 mL of the culture solution was inoculated into 45 mL of a fresh enrichment culture medium and incubated under the same conditions for another 3 days. An enrichment culture solution was obtained by continuously incubating three times with a cycle of three days. The enrichment culture solution was inoculated onto Luria-Bertani (LB) solid culture medium through a streak plate method and incubated at 30 °C in a constant-temperature incubator for 48 hours.

1 mL of the enrichment culture solution was transferred into a test tube containing 9 mL of sterile water to give a mixture. The mixture was thoroughly homogenized by repeated pipetting and vortexing to obtain a 10⁻¹ dilution. Serial dilutions were performed accordingly until a dilution of 10⁻⁷ was achieved. Bengal red agar culture medium was prepared, poured into a plate, cooled and solidified. Subsequently, 100 μL of a corresponding dilution was inoculated onto the Bengal red agar culture medium and spread onto the surface of the plate by using a sterile spreader, with three replicates for each dilution level. The plate was inverted and incubated at 28 °C for 5-7 days. A colony exhibiting rapid growth and favorable morphology was selected and streaked onto a fresh Bengal red agar plate, followed by incubation at 28 °C for 3-5 days. This purification procedure was repeated until a purified strain was obtained. The purified strain from the plate was then inoculated onto a Bengal red agar slant and stored at −18 °C.

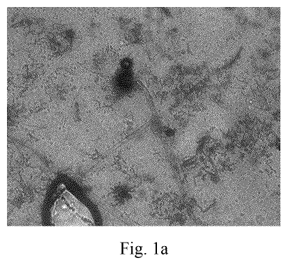

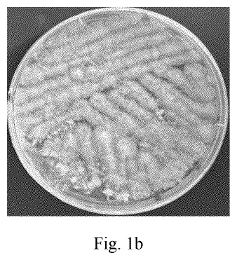

Biochemical, physicochemical, morphological and scanning characterization were performed on the purified strain. The results were shown below. The hyphae were white and cottony in an initial stage, turning yellowish-green to dark green upon maturation due to the production of conidia. Positive results were observed for cellulose hydrolysis, starch hydrolysis, gelatin liquefaction, lipid production and hydrogen peroxide (H₂O₂) test, while acid production was negative. The colony appeared white initially, later becoming yellowish-green, cottony and rapidly expanding (A microscopic photograph and a colonial morphology of Trichoderma longibrachiatum strain 39-2 on Bengal red solid culture medium after 3 days of incubation were shown in FIGS. 1a-1b.).

Genomic deoxyribonucleic acid (DNA) was extracted from the purified strain, and polymerase chain reaction (PCR) amplification was performed by using primers of 27F and 1492R. The PCR products were sequenced by using an ABI 3730 sequencing platform (from Guangdong meige gene technology Co., Ltd.). The resultant sequences were compared against the national center for biotechnology information (NCBI) database, and a phylogenetic tree was constructed by using a neighbor-joining method based on basic local alignment search test (BLAST) results.

The formulations of the culture mediums provided herein were shown below.

(1) Enrichment culture medium (fungi) included 1% (v/v) crude oil, NH₄NO₃ 6 g, K₂HPO₄ 5 g, KH₂PO₄ 5 g, MgSO₄·7H₂O 2 g, CaCl₂) 2 g, nutrient agar 18-20 g and distilled water 1000 mL with a natural pH. 1% Bengal red aqueous solution and 1% streptomycin solution (prepared fresh) were prepared. For each 1000 mL of culture medium, 3.3 mL of the Bengal red solution was added. After the culture medium was melted and cooled to 55-60 °C, 3.3 mL of the streptomycin solution was added to per 100 mL of culture medium.

(2) Bengal red solid culture medium (fungal slant culture medium) was prepared by the following steps. 36.7 g of Bengal red agar culture medium was weighed, added to 1000 mL of distilled water, heated to boiling on an electric hotplate until fully dissolved and then dispensed.

(3) Gelatin liquefaction culture medium included peptone 5 g, gelatin 200 g, glucose 20 g and distilled water 1000 mL.

(4) Starch hydrolysis culture medium included soluble starch 2.0 g, K₂HPO₄ 10 g, KH₂PO₄ 0.3 g, NaCl 0.5 g, MgCO₃ 1 g, KNO₃ 1 g, nutrient agar 15 g and distilled water 1000 mL with a pH of 7.5-7.8.

(5) Cellulose hydrolysis culture medium included MgSO₄·7H₂O 0.5 g, K₂HPO₄ 0.5 g, NaCl 0.5 g, KNO₃ 1 g, a filter paper strip approximately 5 cm in length and distilled water 1000 mL.

(6) Lipid production culture medium included glucose 5 g, and 10% (v/v) bean sprout extract 100 mL. It was dispensed into 50 mL conical flasks, with 20 mL in each flask and sterilized at 115 °C for 20 min.

(7) Acid production culture medium included glucose 5 g, sterilized CaCO₃ 0.5 g, yeast extract 100 mL and nutrient agar 2 g.

The results were shown below.

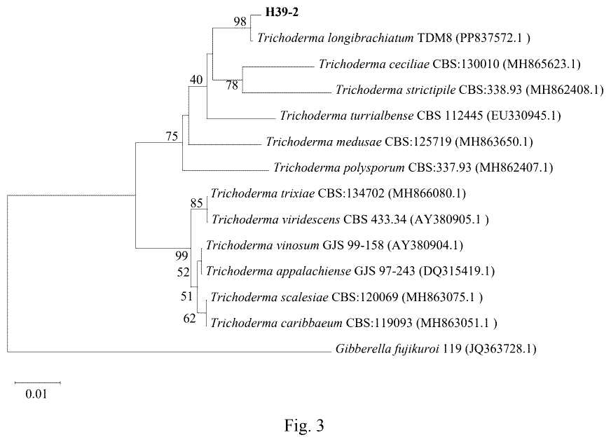

The strain 39-2 involved herein was Trichoderma longibrachiatum strain. Its internal transcribed spacer (ITS) sequence was shown as SEQ ID NO: 1. The strain 39-2 exhibited a 98% similarity to a published strain (U.S. Plant Pat. No. 837,572.1). The strain has been deposited the CGMCC on Sep. 5, 2025 with the accession number of CGMCC No. 42182. The phylogenetic tree was shown in FIG. 3.

The SEQ ID NO: 1 is shown as follows:

TGGAAGTAAAAATCGTAACAAGGTCTCCGTTGGTGAACCAGCGGAGG
GATCATTACCGAGTTTACAACTCCCAAACCCCAATGTGAACGTTACCAAT
CTGTTGCCTCGGCGGGATTCTCTTGCCCCGGGCGCGTCGCAGCCCCGGAT
CCCATGGCGCCCGCCGGAGGACCAACTCCAAACTCTTTTTTCTCTCCGTC
GCGGCTCCCGTCGCGGCTCTGTTTCATTTTTGCTCTGAGCCTTTCTCGGC
GACCCTAGCGGGCGTCTCGAAAATGAATCAAAACTTTCAACAACGGATCT
CTTGGTTCTGGCATCGATGAAGAACGCAGCGAAATGCGATAAGTAATGTG
AATTGCAGAATTCAGTGAATCATCGAATCTTTGAACGCACATTGCGCCCG
CCAGTATTCTGGCGGGCATGCCTGTCCGAGCGTCATTTCAACCCTCGAAC
CCCTCCGGGGGGTCGGCGTTGGGGATCGGCCCCTCACCGGGCCGCCCCCG
AAATACAGTGGCGGTCTCGCCGCAGCCTCTCCTGCGCAGTAGTTTGCACA
CTCGCACCGGGAGCGCGGCGCGGCCACAGCCGTAAAACACCCCAAACTTC
TGAAATGTTGACCTCGGATCAGGTAGGAATACCCGCTGAACTTAAGCATA
TCAATAAGCGGAGGAA.

### Embodiment 2

This embodiment was a crude oil-degrading characterization test of the Trichoderma longibrachiatum strain 39-2, which was performed through the following steps.

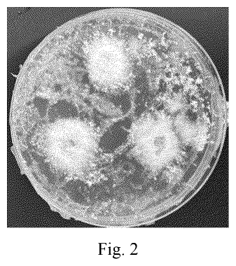

An oily solid culture medium was prepared, sterilized, poured into a plate while hot, cooled naturally and solidified. A diameter of a fungal colony was observed. A colonial morphology of the Trichoderma longibrachiatum strain 39-2 on a fungal petroleum-degrading plate after 7 days of incubation was shown in FIG. 2.

0.9% physiological saline solution (5 mL) was used to rinse filamentous fungal spores of the Trichoderma longibrachiatum strain 39-2 preserved on a slant into 100 mL of Czapek medium, followed by adjusting pH to 7.8 and salinity to 0.9% to obtain a mixture solution. The mixture solution was incubated at 28 °C with shaking at 150 r/min for 36 hours to obtain seed suspension. The seed suspension was then inoculated at 1% (v/v) into a liquid petroleum-degrading culture medium, simultaneously setting up a sterile control group. Their seed suspensions were respectively incubated in a shaker at 28 °C with shaking at 150 r/min for 14 days, and extracted by 20 mL of petroleum ether with a boiling range of 60-90 °C. Their seed suspensions were each extracted three times, and combined to give an extract. Crude oil extracted from a crude oil culture medium without inoculating the aforementioned culture was used as a control group.

The extract was centrifuged for three times to give a supernatant, and the supernatant was collected. The supernatant was dried by anhydrous sodium sulfate (Na₂SO₄), transferred to a volumetric flask and diluted to 20 mL with petroleum ether. After preparing a sample by thoroughly mixing, a 200 μL of the sample was taken and mixed with 100 μL of 0.3 mg/mL C₂₄D₅₀ as an internal standard for later use. The samples were analyzed by gas chromatography-mass spectrometry (GC-MS) using Agilent 6890N gas chromatograph (GC) coupled with a 5973B mass selective detector (MSD). The degradation rates of petroleum hydrocarbon components were calculated based on integrated peak areas from the gas chromatograms, respectively.

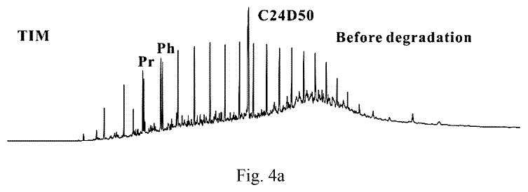

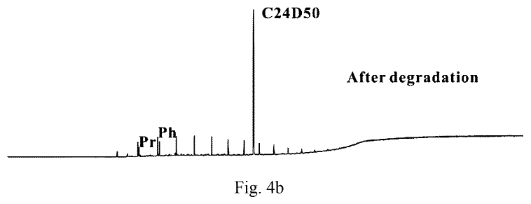

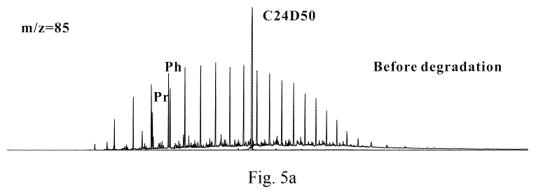

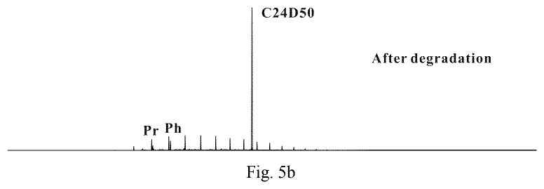

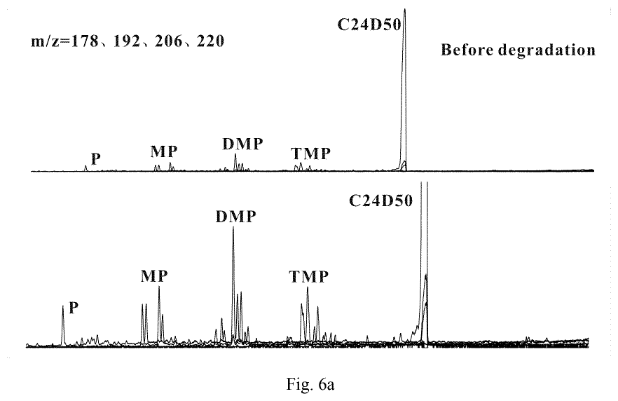

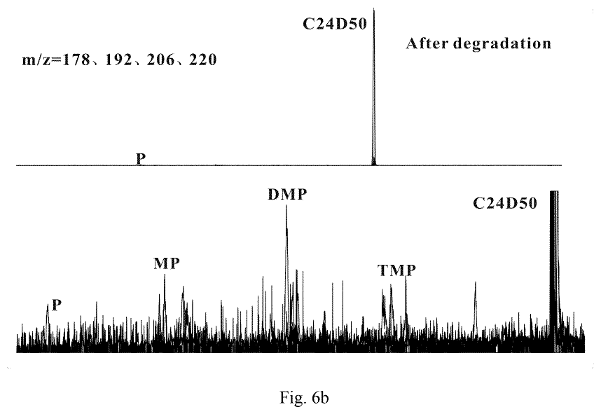

The results were shown in FIGS. 4a-4b. It was calculated that the Trichoderma longibrachiatum strain 39-2 degraded 86.77% of petroleum hydrocarbons within 14 days under an initial crude oil concentration of 2 g/L. The degradation rates of different components were also calculated below. The degradation rate of n-alkanes was 91.5%, while the degradation rates of pristane and phytane in the isoprenoid alkanes were 85.4% and 81.9%, respectively (as shown in FIGS. 5a-5b). Notably, the Trichoderma longibrachiatum strain 39-2 also exhibited higher degradation rates for aromatic hydrocarbon components, with degradation rates of phenanthrene, methylphenanthrene, dimethylphenanthrene and trimethylphenanthrene reaching 97.3%, 91.5%, 93.2% and 93.7%, respectively (as shown in FIGS. 6a-6b). The statistical results of the degradation rates for each component were shown in Table 1.

TABLE 1
Degradation rates of different components of petroleum hydrocarbons

| | | | Control | Experimental | |
|---|---|---|---|---|---|
| | Control | Experimental | Group | Group | Degra- |
| Compo- | Group | Group | Relative | Relative | dation |
| nent | Peak Area | Peak Area | Abundance | Abundance | rate |
| C₂₄D₅₀ | 38646225.92 | 15074479.53 | — | — | — |
| n-alkanes | 46406741.52 | 8543659.11 | 1.200809145 | 0.566763124 | 91.5% |
| Phytane | 1795071.14 | 126940.54 | 0.046448808 | 0.00842089 | 85.4% |
| Pristane | 1030471.59 | 58544.88 | 0.026664223 | 0.003883708 | 81.9% |
| Phenan- | 149602.57 | 2188858.49 | 0.003871078 | 0.145202923 | 97.3% |
| threne | | | | | |
| Methyl- | 563148.5 | 18636.04 | 0.014571889 | 0.001236264 | 91.5% |
| phenan- | | | | | |
| threne | | | | | |
| Dimethyl- | 1088046.31 | 28923.3 | 0.028154012 | 0.001918693 | 93.2% |
| phenan- | | | | | |
| threne | | | | | |
| Trimethyl- | 935488.7 | 22893.2 | 0.02420647 | 0.001518673 | 93.7% |
| phenan- | | | | | |
| threne | | | | | |

In Embodiment 2, the formulations of the culture mediums provided herein were shown below.

(1) Petroleum-degrading culture medium included NH₄NO₃ 2.00 g, MgSO₄·7H₂O 0.20 g, CaCl₂) 0.02 g, FeSO₄ 0.05 g, KH₂PO₄ 0.50 g, K₂HPO₄ 0.50 g and distilled water 1 L and crude oil 1% (v/v) with a pH of 7.8.

(2) Oily solid culture medium was prepared by adding 15-20 g of nutrient agar to the petroleum-degrading culture medium.

(3) Czapek's medium included NaNO₃ 3.00 g, K₂HPO₄ 1.00 g, MgSO₄·7H₂O 0.50 g, KCl 0.50 g, FeSO₄ 0.01 g, sucrose 30.00 g and distilled water 1 L with a pH of 7.8.

In summary, the Trichoderma longibrachiatum strain 39-2 provided herein can degrade petroleum hydrocarbons and exhibit high degradation efficiency for n-alkanes, isoprenoid alkanes and methylphenanthrene series.

Disclosed above are merely preferred embodiments of the disclosure, and are not intended to limit the present disclosure. For those of ordinary skill in the art, any modification, equivalent replacement and improvement can be made within the spirit of the present disclosure, and shall fall within the scope of the disclosure defined by the appended claims.

What is claimed is:

1. A Trichoderma longibrachiatum strain, wherein the Trichoderma longibrachiatum strain has been deposited in China General Microbiological Culture Collection Center (CGMCC) on Sep. 5, 2025 with an accession number of CGMCC No. 42182.

2. A method of degrading petroleum hydrocarbons in a petroleum hydrocarbon-containing sample, comprising:
mixing the Trichoderma longibrachiatum strain of claim 1 with the petroleum hydrocarbon-containing sample.

3. The method of claim 2, wherein the petroleum hydrocarbon-containing sample is crude oil.

4. The method of claim 2, wherein the petroleum hydrocarbons comprise at least one of n-alkanes, isoprenoid alkanes and aromatic hydrocarbons, wherein the isoprenoid alkanes are selected from the group consisting of pristane, phytane and a combination thereof.

5. A culture, wherein the culture is obtained through culture of the Trichoderma longibrachiatum strain of claim 1 in a culture medium.

6. The culture of claim 5, wherein the culture medium has a pH of 7.5-7.8 and a salinity of 0.9%.

7. A method of treating a petroleum hydrocarbon-polluted sample, comprising:
inoculating the Trichoderma longibrachiatum strain of claim 1 into the petroleum hydrocarbon-polluted sample followed by culture.

8. The method of claim 7, wherein the petroleum hydrocarbon-polluted sample is a petroleum-polluted environmental sample.

9. A method of treating a petroleum hydrocarbon-polluted sample, comprising:
inoculating the culture of claim 5 into the petroleum hydrocarbon-polluted sample followed by culture.

10. The method of claim 9, wherein the petroleum hydrocarbon-polluted sample is a petroleum-polluted environmental sample.
